(19) United States
(12) Patent Application Publication
(10) Pub. No.: US 2012/0329860 A1
(43) Pub. Date: Dec. 27, 2012

(54) Uses of Beta-Nicotinamide Adenine Dinucleotide

(75) Inventors: Umapathy N. Siddaramappa, Martinez, GA (US); Alexander D. Verin, Augusta, GA (US); Joyce Gonzales, Augusta, GA (US)

(73) Assignee: MEDICAL COLLEGE OF GEORGIA INSTITUTE, INC, Augusta, GA (US)

(21) Appl. No.: 13/603,913

(22) Filed: Sep. 5, 2012

Related U.S. Application Data
(63) Continuation-in-part of application No. PCT/US2011/000425, filed on Mar. 7, 2011.
Application No. PCT/US2011/000425 is a continuation of application No. 61/339,565, filed on Mar. 5, 2010.

(51) Int. Cl. A61K 31/7084; A61P 11/06; A61P 35/00; A61P 11/08; A61P 11/00; A61P 29/00; A61P 31/00

(52) U.S. Cl. 514/44 R

(57) ABSTRACT

The present invention provides methods for treating inflammation in the lungs of a subject in need of such treatment, comprising the step of administering an effective amount of a composition comprising b-nicotinamide adenine dinucleotide to the subject. Also provided is a method of increasing integrity of a vascular barrier in a subject, comprising the step of contacting one or both of human P2Y1 receptors or P2Y11 receptors in the subject with an amount of a composition comprising beta-nicotinamide adenine dinucleotide effective to activate the receptors; wherein activation thereof increases the integrity of the vascular barrier in the subject.

### CROSS-REFERENCE TO RELATED APPLICATIONS

This continuation-in-part application claims benefit of priority under U.S.C. §120 of international application PCT/US2011/000425, filed Mar. 7, 2011, which claims benefit of priority under 35 U.S.C. §119(e) of provisional application U.S. Ser. No. 61/339,565, filed Mar. 5, 2010, now abandoned, the contents of both of which hereby are incorporated by reference.

### FEDERAL FUNDING LEGEND

This invention was made with government support under Grants HL083327 and HL67307 awarded by the National Heart, Lung, and Blood Institute. The government has certain rights in this invention.

### BACKGROUND OF THE INVENTION

1. Field of the Invention

The present invention relates to the fields of pulmonology and treatment of lung disorders. More specifically, the present invention relates to, inter alia, methods for using b-nicotinamide adenine dinucleotide in the treatment of various pulmonary diseases or disorders.

2. Description of the Related Art

The vascular endothelium is a semi-selective diffusion barrier between the plasma and interstitial fluid and is critical to vessel wall homeostasis. Inflammatory factor-induced barrier dysfunction of the endothelium is associated with cytoskeletal remodeling, disruption of cell-cell contacts and the formation of paracellular gaps. Reorganization of the endothelial cytoskeleton leads to alteration in cell shape and provides a structural basis for increase of vascular permeability, which has been implicated in the pathogenesis of diseases. Disruption of the endothelial barrier occurs during inflammatory diseases such as acute lung injury (ALI) and acute respiratory distress syndrome (ARDS), with an overall mortality rate of 30-40%, and results in the movement of fluid and macromolecules into the interstitium and pulmonary air spaces causing pulmonary edema.

Endothelial cells (EC) are connected to each other by a complex set of junctional proteins that comprise tight junctions (TJs), adherent junctions (AJs) and gap junctions (GJs).

Endothelial adherent junctions contain vascular endothelial (VE-cadherin) as the major structural protein responsible for homophilic binding and adhesion of adjacent cells. VE-cadherin is essential for proper assembly of adherent junctions and development of normal endothelial barrier function. Ectopic expression of a cadherin mutant lacking VE-cadherin extracellular domain in dermal endothelial cells resulted in a leaky junctional barrier indicating the significance of VE-cadherin. Although the precise mechanisms of the regulation of junctional assembly by VE-cadherin have not been identified, actin-binding proteins appear to be crucial. Lampugnani et al showed that transfection of VE-cadherin cDNA in endothelial cells from VE-cadherin-null murine embryos induced actin cytoskeleton rearrangement and activated Rho family GTPase Rac1. Likewise, engagement of VE-cadherin activates Rac1 suggesting a role of VE-cadherin in recruiting Rac1 during cytoskeletal reorganization.

During vascular injury, lysed cells are a source of extracellular nucleotides. Additionally, vascular endothelial cells are also regulated by extracellular nucleotides released from platelets. Beta-nicotinamide adenine dinucleotide is a coenzyme found in all living cells. In metabolism, b-NAD is involved in redox reactions, carrying electrons from one reaction to another and these electron transfer reactions are the main known function of b-NAD. It is also used in other cellular processes, notably as a substrate of enzymes that add or remove chemical groups from proteins, and in posttranslational modifications. b-NAD is a cytokine targeting human polymorphonuclear granulocytes and a rapid increase of cAMP was observed followed by exposure to extracellular b-NAD. Present in nanomolar to sub-micromolar concentrations in human serum, b-NAD, released extracellularly from the injured cells, could be involved in various signaling mechanisms. b-NAD is an agonist of human P2Y1 and P2Y11 receptors, respectively.

ARDS and ALI are commonly seen in Intensive Care Units with a mortality rate of 15-40%. Common contributory conditions include sepsis, septic shock and pneumonia. The acute phase of ALI/ARDS is characterized by the influx of protein-rich edema fluid into the air spaces as a consequence of increased permeability of the alveolar capillary barrier. Pulmonary endothelial cell barrier breakdown is one of the hallmarks of these lung diseases. In spite of intense research, there is still no successful pharmacologic treatment strategy for lung diseases involving pulmonary endothelial cell barrier breakdown although surfactant, inhaled nitric oxide, corticosteroids, antifungal drugs and phosphodiesterase inhibitors have been used unsuccessfully. The untreated manifestations are pulmonary edema, hypoxemia and heterogeneous parenchymal consolidation.

Thus, there is a continued need in the art for improved methods to treat lung diseases involving pulmonary endothelial cell barrier breakdown. The present invention fulfills this longstanding need and desire in the art.

### SUMMARY OF THE INVENTION

The present invention is directed to a method for treating inflammation in the lungs of a subject in need of such treatment, comprising the step of: administering an effective amount of a composition comprising b-NAD to the subject.

In another embodiment, the present invention provides a method for treating a pulmonary disorder in a subject in need of such treatment, comprising the steps of administering an effective amount of a composition comprising b-NAD to the subject, wherein administration of said composition results in an average minimum plasma concentration b-NAD that is greater than 100 mM in the plasma of the subject and an average maximum concentration of b-NAD that is less than 100 mM in the plasma of the subject; and administering a therapeutic agent selected from the list consisting of an anti-inflammatory agent, bronchodilator and an antibiotic.

In yet another embodiment, the present invention provides a method for increasing integrity of a vascular barrier in a subject, comprising the step of contacting one or both of human P2Y1 receptors or P2Y11 receptors in the subject with an amount of a composition comprising beta-nicotinamide adenine dinucleotide effective to activate said receptors; wherein activation thereof increases the integrity of the vascular barrier in the subject.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1C show that the extracellular b-NAD enhances barrier function of human pulmonary artery endothelial cells (HPAEC) monolayers and increases VE-cadherin presentation in cell-cell contacts. FIG. 1A: Dose-dependent TER response of b-NAD. HPAEC were challenged with increasing concentration of b-NAD (10-100 mM). Data are representative of several independent experiments (minimum of three) (*p<0.05 compared with control). FIG. 1B: Immunofluorescence staining of VE-cadherin in quiescent and b-NAD-stimulated HPAEC monolayers. The cells were treated with 50 μM b-NAD for 30 minutes, then fixed and immunostained using VE-cadherin antibody. Appreciably more VE-cadherin was recruited to the area of cell-cell junctions after b-NAD treatment. Arrows indicate overlapping edges of neighboring cells. FIG. 1C: Quantification of the surface area of the cell-cell interface. The percentage of total cell-surface area occupied by VE-cadherin-positive cell-cell junctions was calculated for 20 cells in each group. b-NAD induced a significant increase in cell-cell interface surface area as a percentage of total cell surface area (*p<0.05 compared with control). The box and Whiskers plot show the means (lines at the box centers, 17.42% and 61.03% for control and b-NAD-treated cells respectively).

FIGS. 2A-2B show the expression of b-NAD-activated purine receptors P2Y1 and P2Y11 on mRNA and protein levels in HPAEC. FIG. 2A: The receptor mRNA expressions were examined by Real-Time RT-PCR. Data were calculated relative to internal housekeeping gene (18S rRNA) and are expressed as fold change compared to control ±SEM (n=4). FIG. 2B: The cell lysates obtained from HPAEC were analyzed by SDS-PAGE followed by immunoblotting using rabbit polyclonal antibodies against P2Y1 and P2Y11. Position of 40 kDa protein marker is shown by arrow. Immunoblotting of b-actin was used as a loading control.

FIGS. 3A-3D show that the inhibitory analysis (selective antagonists and siRNA-mediated depletion) of the involvement of P2Y1 and P2Y11 receptors in b-NAD-stimulated TER increase. FIG. 3A: HPAEC were pretreated with either 10 mM MRS2279 (P2Y1 antagonist) or 1 mM NF157 (P2Y11 antagonist) for 30 min prior b-NAD stimulation and used in ECIS assay. (*p<0.05 compared with control). FIG. 3B: RT-PCR analysis of the expression of P2Y1 and P2Y11 mRNAs in the cells treated with scrambled and receptor-specific siRNA. siRNA approach was proved to be very effective in the depletion of P2Y1 and P2Y11 expression. Expression of hypoxanthine-guanine phosphoribosyltransferase (HPRT) was used as a loading control. Depletions of P2Y11 (FIG. 3C) or P2Y1 (FIG. 3D) receptors negatively affect b-NAD-mediated TER response. HPAEC plated in ECIS arrays were transfected with respective scrambled (nsRNA) and siRNA. Fourty-eight hrs after transfection, the cells were used in ECIS assay in the presence or absence of b-NAD. Time-points of b-NAD addition are indicated by arrows. (*p<0.05 compared with control).

FIGS. 4A-4C show that extracellular b-NAD protects HPAEC monolayers from barrier-disruptive effects of thrombin and Gram-negative and Gram-positive bacterial toxins, lipopolysaccharide (LPS) and pneumolysin (PLY). HPAEC plated in ECIS arrays were challenged with either 100 nM thrombin (FIG. 4A) or 100 mM LPS (FIGS. 4B) or 31.2 ng/ml PLY (FIGS. 4C). The challengers were added either alone or in mixture with 50 mM b-NAD. b-NAD consistently prevented HPAEC barrier dysfunction in the cells treated with thrombin or PLY and significantly protected barrier integrity in the cells treated with LPS. (*p<0.05 compared with control).

FIGS. 5A-5C show the effect of cytoskeletal alterations on b-NAD-induced endothelial cell barrier protection. FIG. 5A: HPAEC were pretreated with either vehicle or cytochalasin B for 30 minutes and then stimulated with 50 μM β-NAD in TER measurement assay. Actin depolymerization decreased TER and completely prevented the effect of β-NAD on TER. FIG. 5B: HPAEC were pretreated with either vehicle or the microtubule-disrupting agent, nocodazole, for 30 min and then stimulated with 50 μM β-NAD. Disruption of microtubules decreased TER, but failed to alter β-NAD-induced increase in TER. Results are presented as mean±SE and derived from three independent experiments (*p<0.05 compared with control). FIG. 5C: β-NAD treatment decreases myosin light chain (MLC) phosphorylation stimulated by LPS. HPAEC treated by either vehicle or LPS alone, or LPS/β-NAD mixture for 4 hrs were lysed and analyzed by SDS-PAGE followed by immunoblotting with anti-phosphoMLC antibody. Immunoblotting with anti-MLC antibody was used as a loading control. Data obtained indicate that barrier-protective mechanism of β-NAD may be realized via stimulation of MLC phosphatase activity, decreasing phosphoMLC levels and preventing, therefore, actin stress fiber formation.

FIGS. 6A-6D show molecular mechanisms of β-NAD-activated endothelial barrier enhancement in HPAEC. FIG. 6A: β-NAD treatment activated cAMP-dependent PKA. HPAEC were pretreated with either vehicle or PKA-specific inhibitor, 5 mM H-89, for 30 min and then stimulated with 50 μM β-NAD in TER measurement assay. The inhibitor pretreatment significantly attenuated β-NAD-dependent increase in TER. FIG. 6B: EPAC1 was successfully depleted by siRNA. FIG. 6C: cAMP-activated EPAC1 is involved in β-NAD-activated TER response. HPAEC plated in ECIS arrays were transfected with either scrambled (nsRNA) or EPAC1-specific siRNA. The cells were stimulated with 50 μM β-NAD in TER measurement assay. Successful depletion of EPAC1 led to a significant decrease of β-NAD-activated TER response. FIG. 6D: Downstream target of PKA/EPAC1 pathways, Rac1, is activated in HPAEC upon β-NAD stimulation. The cells treated with 50 mM β-NAD for time periods indicated were used in G-LISA assay to estimate the levels of activated Rac1. Data obtained demonstrate a rapid and dramatic elevation of Rac1-activity in β-NAD -stimulated cells. (*p<0.05 compared with control).

FIG. 7 shows the effect of LPS and β-NAD on pulmonary vascular leak. β-NAD treatment reduced total protein accumulation in the BAL fluid of LPS-induced lung injury. The asterisk indicates that a value significantly (p<0.05) differs from the vehicle group.

FIGS. 8A-8B: shows BAL EBD Extravasations. FIG. 8A, β-NAD reduced total EBA extravasation into lungs of LPS challenged mice. EBD was injected into the internal jugular vein 120 min before the termination of the experiment. LPS challenge increased EBD leakage from the vascular space into surrounding lung tissue in the LPS group with notable attenuation in the LPS/β-NAD mice group. Both groups are compared to control and β-NAD only treated mice. FIG. 8B: b-NAD attenuated EBD leakage into the lung parenchyma on gross examination. Mice were grouped and LPS group received LPS (0.9 mg/kg, i.t.) with PBS (i.v.), LPS/β-NAD group with LPS (0.9 mg/kg, i.t.) and b-NAD (5.46 mg/kg, i.t.), and control group with PBS (12 μl i.t. and 30 μl i.v.). EBD was injected into the right internal jugular vein 2 hr prior to termination of the experiment. The mice were sacrificed at 24 hr and immediately the lungs were flushed with EDTA, harvested, and photographed. Gross observation of the lung at 24 hr showed that the LPS/PBS lung exposure shows increased penetration of the EBD in the lung parenchyma, with minimal leakage in the LPS/b-NAD treated specimen and none visible in vehicle. The asterisk indicates that a value significantly (p<0.05) differs from the vehicle group.

FIG. 9 shows BAL Cells Count, β-NAD reduces WBC accumulation in BAL of LPS treated mice. BAL was collected at 24 hr after treatment, centrifuged, and the cells were counted in hemocytometer. The β-NAD reduced total WBCs in BAL fluid. The asterisk indicates that a value significantly (p<0.05) differs from the vehicle group.

FIG. 10 shows that β-NAD inhibits the inflammation in lungs of mice in LPS-induced ALI. Lungs perfused free of blood after perfusion with EDTA, were immersed in 5% buffered paraformaldehyde at 4° C. for 18 h prior to histological evaluation by hematoxylin and eosin staining. H&E staining was done by deparafinizing and hydrating the slides to water. The slides were stained in Harris Hematoxylin for 15 min and Eosin for 30 sec. The slides were dehydrated, cleared and mounted with cytoseal. Histological analysis of the lung tissue obtained from the control mice exposed to PBS showed minimal infiltration of neutrophils. In contrast, mice exposed to LPS (20 mg/kg, i.p.) for 18 h produced prominent neutrophil infiltration and that was attenuated in LPS/β-NAD simultaneously.

FIG. 11 shows that β-NAD decreases the LPS-induced mortality in mice. Mice were injected with β-NAD (100 mg/kg, i.p) or vehicle (saline, i.p) 10 min after LPS (20 mg/kg, i.p) challenge. As a treatment strategy, β-NAD was given twice a day (i.p) for 4 days. Survival rates were recorded for 4 days. *p<0.05 vs the LPS group.

FIG. 12 shows that b-NAD attenuates the LPS-stimulated inflammation. Quantitative real-time PCR analysis of pro-inflammatory/anti-inflammatory cytokines gene expression from lungs of mice challenged with PBS, LPS, and LPS/b-NAD. The Bar represents the average fold change compared with control (PBS) and the expression levels were normalized to the value of housekeeping gene GAPDH mRNA. The asterisk indicates that a value significantly (p<0.05 vs. LPS) differs from the LPS group.

FIG. 13 shows Myeloperoxidase (MPO) activity in mice lungs. Myeloperoxidase activity was determined using a myeloperoxidase assay kit (Cayman Cat # 700160) according to the manufacturer's protocol. LPS significantly elevated in lungs challenged with LPS and the β-NAD treatment attenuated the LPS-induced myeloperoxidase. β-NAD alone has no effect. Data are expressed as means±SE (n=6 in each group).

FIG. 14 shows histological assessment. Paraffin sections of sham and LPS exposed wild type mice were stained for myeloperoxidase. LPS significantly increased the neutrophil infiltration into the lungs as assessed by the myeloperoxidase staining and the β-NAD treatment attenuated the LPS-induced myeloperoxidase.

### DETAILED DESCRIPTION OF THE INVENTION

The present invention is directed to a method for treating inflammation in the lungs of a subject in need of such treatment, comprising the step of administering an effective amount of a composition comprising beta-nicotinamide adenine dinucleotide or β-NAD to the subject. This method is useful in treating inflammation induced by or associated with elevated levels of one or more cytokines in the lungs. Representative examples of such cytokines include but are not limited to interleukin-1alpha, interleukin-1beta, interleukin-8, interleukin-17, TNF-alpha, interferon-gamma and tissue growth factor beta. Furthermore, such lung inflammation may be induced by or associated with increased levels of one or more inflammatory cells in the lungs. Representative examples of such inflammatory cells include but are not limited to eosinophils, lymphocytes, macrophages, neutrophils and monocytes.

Generally, the inflammation is associated with asthma, allergic asthma, infection, emphysema, inflammatory lung injury, bronchiolitis obliterans, pulmonary sarcoisosis, chronic obstructive pulmonary disease, interstitial lung disease, idiopathic pulmonary fibrosis, adult respiratory distress syndrome, bronchiectasis, lung eosinophilia, interstitial fibrosis, acute lung injury, sepsis, cystic fibrosis, transplantation of an organ, tissue and/or cells to the subject. Typically, administration of the composition elevates levels of an anti-inflammatory cytokine in the lungs of the subject. Representative examples of such anti-inflammatory cytokines are interleukin-4, interleukin-13 and interleukin-10. Typically, administration of this composition reduces levels of a pro-inflammatory cytokine in the lungs of the subject. Representative examples of such pro-inflammatory cytokines are interleukin-1alpha, interleukin-1beta, interleukin-8, interleukin 17, TNF-alpha, interferon-gamma and tissue growth factor-beta.

Preferably, administration of the composition results in an average minimum plasma b-NAD concentration of greater than 100 mM in the plasma of the subject and an average maximum b-NAD concentration of less than 100 mM in the plasma of the subject. Generally, the composition is administered in a dose of from about 0.1 mg/kg to about 50 mg/kg of the subject's body weight. The composition may be administered by any acceptable route, including but not limited to systemic, oral, intravenous, intramuscular, subcutaneous, intraorbital, intranasal, intracapsular, intraperitoneal, intracisternal, intratracheal, intraarticular administration, or by absorption through the skin, and aerosol administration. A person having ordinary skill in this art would readily recognize that the composition of the present invention may be combined with other therapeutically effective agents, including but not limited to an anti-inflammatory agent, bronchodilator and an antibiotic.

The present invention is further directed to a method for treating a pulmonary disorder in a subject in need of such treatment, comprising the steps of administering an effective amount of a composition comprising β-NAD to the subject, wherein administration of the composition result in an average minimum plasma concentration of β-NAD that is greater than 100 mM in the plasma of the subject and an average maximum concentration of β-NAD is less than 100 mM in the plasma of the subject; and administering a therapeutic agent selected from the list consisting of an anti-inflammatory agent, bronchodilator and an antibiotic. Typically, the administration of the composition elevates levels of an anti-inflammatory cytokine in the lungs of the subject, reduces levels of a pro-inflammatory cytokine in the lungs of the subject, or elevates levels of an anti-inflammatory cytokine and reduces levels of a pro-inflammatory cytokine in the lungs of the subject.

The present invention is further directed to a method for increasing integrity of a vascular barrier in a subject, comprising the step of contacting one or both of human P2Y1 receptors or P2Y11 receptors in the subject with an amount of a composition comprising beta-nicotinamide adenine dinucleotide effective to activate the receptors; wherein activation thereof increases the integrity of the vascular barrier in the subject. Typically, a pulmonary disorder in the subject has reduced the integrity of the vascular barrier. Generally, the pulmonary disorder is asthma, allergic asthma, infection, emphysema, inflammatory lung injury, bronchiolitis obliterans, pulmonary sarcoisosis, chronic obstructive pulmonary disease, interstitial lung disease, idiopathic pulmonary fibrosis, adult respiratory distress syndrome, bronchiectasis, lung eosinophilia, interstitial fibrosis, acute lung injury, sepsis, inflammation mediated lung cancer, or cystic fibrosis.

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device or method described herein can be implemented with respect to any other device or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “contacting” refers to any suitable method of bringing a compound or a composition into contact with a cell. In vitro or ex vivo this is achieved by exposing the cell to the compound or agent in a suitable medium. For in vivo applications, any known method of administration is suitable as described herein.

As used herein, the term “subject” refers to any human or non-human recipient of the composition described herein.

The following example(s) are given for the purpose of illustrating various embodiments of the invention and are not meant to limit the present invention in any fashion.

### EXAMPLE 1

### Materials

Reagents were obtained from Sigma-Aldrich (St. Louis, Mo.) unless otherwise indicated. Mouse monoclonal VE-cadherin antibody was purchased from BD Biosciences (San Diego, Calif.). Rabbit polyclonal antibodies against P2Y1 and P2Y11 receptors were obtained from Santa Cruz Biotechnology (Santa Cruz, Calif.). siPORT Amine transfection reagent was obtained from Ambion (Austin, Tex.). P2Y1, P2Y11-, EPAC1- and Rac1-specific siRNAs were purchased from Santa Cruz Biotechnology. TRIzol was obtained from Invitrogen (Carlsbad, Calif.). P2Y1- and P2Y11-specific antagonists were obtained from Tocris (Ellisville, Mo.). PKA inhibitor, H89, was purchased from Calbiochem (San Diego, Calif.). Phospho-MLC-specific antibodies were purchased from Cell Signaling (Beverly, Mass.). G-LISA kit was obtained from Cytoskeleton Inc. (Denver, Colo.).

### Cell Culture

Human pulmonary artery endothial cells (HPAEC) and EBM-2 complete medium were purchased from Lonza (Allendale, N.J.). HPAEC were cultured according to the manufacturer's protocol and utilized at early (3-6) passages.

### Measurement of Endothelial Monolayer Electrical Resistance

The barrier properties of endothelial cells monolayers were characterized using a highly sensitive electrical cell-substrate impedance sensing (ECIS) instrument to measure transendothelial electrical resistance (TER) as described (Birukova et al., 2004a. Microvasc Res 67(1):64-77; Kolosova et al., 2005, Circ Res 97(2):115-124). The TER data was normalized to the initial voltage.

### Immunofluorescence Microscopy

Immunostaining was performed as described (Kolosova et al., 2005, Circ Res 97(2):115-124). The DNA-binding, fluorescent dye 7-amino-actinomycin D (7AAD) was used to stain cell nuclei. The percentage of total cell surface area occupied by VE-cadherin-positive cell-cell junctions was quantitatively determined using Zeiss Microscope quantification Software.

### Semi-Quantitative RT-PCR Analysis

To compare the amounts of P2Y1 and P2Y11 mRNAs, the total RNA (1.0 μg) isolated from HPAEC was subjected to PCR in 25-μl reaction mixture using reagents from Superscript One Step RT-PCR kit (Invitrogen, Carlsbad, Calif.). 18S ribosomal RNA 184 by fragment (internal control for normalization) was amplified using 50 nM primers from TaqMan Gold RT-PCR Core Reagents Kit (Applied Biosystems, Foster City, Calif.). To amplify a 134 by fragment of Homo sapiens P2Y1 cDNA (Accession No. NM_{—}002563.2), the primers used were: forward, 5′-TATTCATCATCGGCTTCCTGGGCA-3′ (SEQ ID NO: 1); reverse, 5′-AGCGGCATCTCCGTGTACATGTTCAA-3′ (SEQ ID NO: 2); and probe, 5′-AGCGGCATCTCCGTGTACATGTTCAA-3′. For the amplification of 189 by fragment of Homo sapiens P2Y11 cDNA (Accession No. NM_{—}002566.4), the following primers were used: forward, 5′-CTCCTATGTGCCCTACCACATCA-3′ (SEQ ID NO: 3); reverse, 5′-AGCTTTGCAGACATAGCCCAGGCCA-3′ (SEQ ID NO: 4); and probe, 5′-AGCTTTGCAGACATAGCCCAGGCCA-3′. For the amplification of 391 by fragment of Homo sapiens EPAC1 (Accession No. NM_{—}001098351), the following primers were used: forward, 5′-TTGTTGTCAACCCACACGAAGTGC-3′ (SEQ ID NO: 5); reverse, 5′-GAGGCCAAACATGACGGCAAAGAA-3′ (SEQ ID NO: 6). The final concentration of all primers used was 200 nM. The PCR products were analyzed by agarose gel electrophoresis.

RT-PCR Analysis of Expression of mRNA Transcripts

The presence of specific mRNA transcripts for P2Y1, P2Y11, and EPAC1 was evaluated by RT-PCR. Total RNA was prepared from HPAEC using TRIzol. For RT-PCR analysis, 1 μg total RNA was reverse transcribed using a RNA-PCR kit (Gene-Amp; Applied Biosystems, Foster City, Calif.) according to the manufacturer's protocol. PCR was performed using 1.0 μmol each of sense and antisense primers, 2.5 U of AmpliTaq DNA polymerase (Applied Biosystems), and the following cycling conditions: 94° C. for 0.5 minutes; 35 cycles of 94° C. for 1 minute, 60° C. for 1 minute, and 72° C. for 1 minute; 1 cycle of 72° C. for 5 minutes. The PCR products were analyzed by agarose gel electrophoresis.

### P2Y1 and P2Y11 Receptor Antagonists Study

HPAEC were pretreated with receptor-specific antagonists, MRS2279 or NF157, for 30 min, and then challenged with 50 mM b-NAD. TER was registered throughout to examine the barrier enhancement induced by b-NAD in the presence or absence of the antagonists.

Depletion of Endogenous mRNA using siRNA Approach

To deplete the mRNA content of endogenous P2Y1, P2Y11 or EPAC1, the cells were treated with respective siRNA duplexes, which guide sequence-specific degradation of the homologous mRNA. A non-specific, scrambled siRNAs were used as a control treatment. HPAEC were plated on 60-mm dishes to yield 60-70% confluence, and transfection of siRNAs was performed using siPORT Amine transfection reagent according to the manufacturer's protocol. Briefly, cells were serum-starved for 1 hr followed by incubation with 20 nM of target siRNA (or scrambled siRNA) for 6 hrs in serum-free media. Then media with serum was added (1% serum final concentration) for 42 hrs before biochemical experiments, ECIS and/or functional assays were conducted. To estimate the efficiency of mRNA depletion, 48 hrs later, the cells were lysed in TRIzol and specific mRNA depletion was analyzed by RT-PCR. For TER measurement, cells were plated to yield 60-70% confluence in electrode wells and transfected with siRNA as described (Kolosova et al., 2005, Circ Res 97(2):115-124).

### Immunoblotting and G-LISA

Protein extracts were separated by SDS-PAGE, transferred to nitrocellulose membrane and probed with specific antibodies. Horseradish peroxidase-conjugated goat anti-rabbit IgG antibody (Santa Cruz Biotechnology, Santa Cruz, Calif.) was used as the secondary antibody, and immunoreactive proteins were detected using enhanced chemiluminescence detection kit (ECL) according to the manufacturer's protocol (Amersham, Little Chalfont, UK). For quantification, immunoblot data were analyzed using NIH Image 1.63 software. Active Rac1 was determined using G-LISA Rac activation assay according to the manufacturer's recommendations (Cytoskeleton Inc., Denver, Colo.).

### Statistical Analysis

All measurements are presented as the mean±SEM of at least 3 independent experiments. To compare results between groups, a 2-sample Student t test was used. For comparison among groups, 1-way ANOVA was performed. Differences were considered statistically significant at p<0.05.

### Animals

Female C57BL/6J mice (8-10 weeks old) weighing 20-25 g were purchased from Charles River Laboratory (Wilmington, Mass.). Animals were housed in plastic cages and had access to food and water. The animals were kept at room temperature and exposed to continuous cycles of 12 hr light and darkness.

### Animal Surgical Procedure

Mice were anesthetized with ketamine (150 mg/kg) and acetylpromazine (15 mg/kg) intraperitoneally (i.p.) before the exposure of the trachea via neck incision and intubation with 20-guage catheter and the right internal jugular vein was exposed via right chest incision for PBS or b-NAD installation. The mice were randomly divided into groups. LPS or sterile saline was instilled intratracheally (i.t.) via a 20-gauge catheter. Simultaneously, mice received either b-NAD (5.4 mg/kg, equivalent to final calculated plasma concentration 50 mM or PBS in the control group intravenously (i.v.) through the internal jugular vein (IJ). The animals were allowed to recover for 18 hr. EBD was given through the IJ 2 hr prior to termination of the experiment. At termination, bronchoalveolar lavage (BAL) was collected. BAL was 1ml of 10% HBSS through the endotracheal catheter immediately on sacrifice with aspiration. BAL was immediately centrifuged and processed. After the BAL, ethylenediaminetetraacetic acid (EDTA) was used to flush the lungs of blood via the right heart ventricle and the lungs were then harvested. BAL and lungs were collected and stored at −70° C. for evaluation of lung injury.

Protein Estimation and Cell Count from the BAL

The BAL was centrifuged (500 g, 15 min, 4° C.), supernatant was centrifuged again (16,500 g, 10 min, 4° C.), and pure BAL fluid was used to measure total protein (BCA Protein Assay kit; Pierce Chemical, Rockford, Ill.). Cell pellets were suspended in Hanks' solution, and red blood cells were lysed by hypotonic shock (0.2% NaCl) for 5 min. Cell suspensions were centrifuged (500 g, 10 min, 4° C.). Then formalin (3.7%) was instilled onto the cell pellet and the cells were then counted on a hemocytometer.

### Lung Permeability Measurements Using Evans Blue Dye-Albumin (EBD)

Measurement of EBD concentration in the lungs was performed by injection of EBD (20 mg/kg) into the right internal jugular vein 2 hr before the termination of the experiment to assess the vascular leak. Lungs free of blood were weighed and snap frozen in liquid nitrogen. The left lung was weighed and homogenized, then incubated with two volumes of formamide (18 hr, 60° C.) and centrifuged (5000 g, 30 min, 20° C.). The extravasated EBD concentration (mg/g, lung) in the lung homogenate was calculated against a standard curve. In a separate experiment the EBD was injected into the right internal jugular vein as described above at 2 hrs prior to termination of the experiment and the left lung gross anatomy view was photographed with a Leica NCL150 Camera.

### Lung Histology

Lungs perfused free of blood after perfusion with EDTA, were immersed in 5% buffered paraformaldehyde for 18 hr at 4° C. prior to histological evaluation by hematoxylin and eosin staining (H&E staining). The right lung lobes were used for consistency. H&E staining was done by deparafinizing and hydrating the slides to water. The slides were stained in Harris Hematoxylin for 15 min and Eosin for 30 sec. The slides were dehydrated, cleared and mounted with cytoseal.

Quantitative Real-Time Polymerase Chain Reaction (qPCR)

Total RNA was prepared from the lung of mouse tissue using RNeasy mini kit (Qiagen, Valencia, Calif.). The mRNA was reverse-transcribed into complementary deoxyribonucleic acid (cDNA) using iScript reagents from Bio-Rad on a programmable thermal cycler (PCR-Sprint, Thermo Electron, Milford, Mass.). 50 ng of cDNA was amplified in each real-time polymerase chain reaction using ABgene reagents (distributed by Fisher Scientific), Bio-Rad myiQ Cycler and Custom-designed primers for genes specific to the mice (Integrated DNA Technologies, Coralville, Iowa). The forward and reverse primers sequences are shown in Table 1. The Reverse transcription reaction was carried out for 25 min at 42° C. and terminated for 5 min at 85° C. Real time PCR was performed by denaturation for 30 sec at 94° C., annealing for 30 sec at 60° C. for a total of 40 cycles. The glyceraldehyde-3-phosphate dehydrogenase (GAPDH) was used to normalize the expression of the target genes.

TABLE 1
Primers sequences used in qPCR

| Name | | SEQ | |
|---|---|---|---|
| of | | ID | Accession |
| Gene | Primer sequence | NO | Number |
| GAPDH | F-CATGGCCTCCAAGGAGTAAGA | 7 | M32599 |
| | R-GAGGGAGATGCTCAGTGTTGG | 8 | |
| IL-1α | F-GCACCTTACACCTACCAGAGT | 9 | NM_010554.4 |
| | R-AAACTTCTGCCTGACGAGCTT | 10 | |
| IL-1β | F-GCAACTGTTCCTGAACTCAACT | 11 | NM_008361.3 |
| | R-ATCTTTTGGGGTCCGTCAACT | 12 | |
| IL-4 | F-GGTCTCAACCCCCAGCTAGT | 13 | NM_021283.2 |
| | R-GCCGATGATCTCTCTCAAGTGAT | 14 | |
| IL-6 | F-TAGTCCTTCCTACCCCAATTTCC | 15 | NM_031168.1 |
| | R-TTGGTCCTTAGCCACTCCTTC | 16 | |
| IL-10 | F-GCTCTTACTGACTGGCATGAG | 17 | NM_010548.1 |
| | R-CGCAGCTCTAGGAGCATGTG | 18 | |
| IL-13 | F-CCTGGCTCTTGCTTGCCTT | 19 | NM_008355.3 |
| | R-GGTCTTGTGTGATGTTGCTCA | 20 | |
| IFN-γ | F-ATGAACGCTACACACTGCATC | 21 | NM_008336 |
| | R-CCATCCTTTTGCCAGTTCCTC | 22 | |
| TNF-α | F-CCCTCACACTCAGATCATCTTCT | 23 | NM_013693 |
| | R-GCTACGACGTGGGCTACAG | 24 | |

### Measurement of MPO Activity and Staining

MPO is a hemoprotein that is abundantly expressed in polymorphonuclear leukocytes (neutrophils) and secreted during their activation. MPO assay was carried out according to the manufacturer's assay protocol (Cayman Chemicals). Lungs of vehicle, LPS or LPS/b-NAD treated mice were used for the MPO assay. Lungs (free of blood) were weighed and snap frozen in liquid nitrogen. The left lung was weighed and homogenized in the lysis buffer and then centrifuged (12,000×g, 30 min). The protein from the clear supernatant was estimated and normalized from different treatment and analyzed for the MPO levels by ELISA. Values represent mean±SEM (n=3).

### EXAMPLE 2

### Extracellular β-NAD Increases Transendothelial Electrical Resistance and Affects Endothelial Cell-Cell Junctions

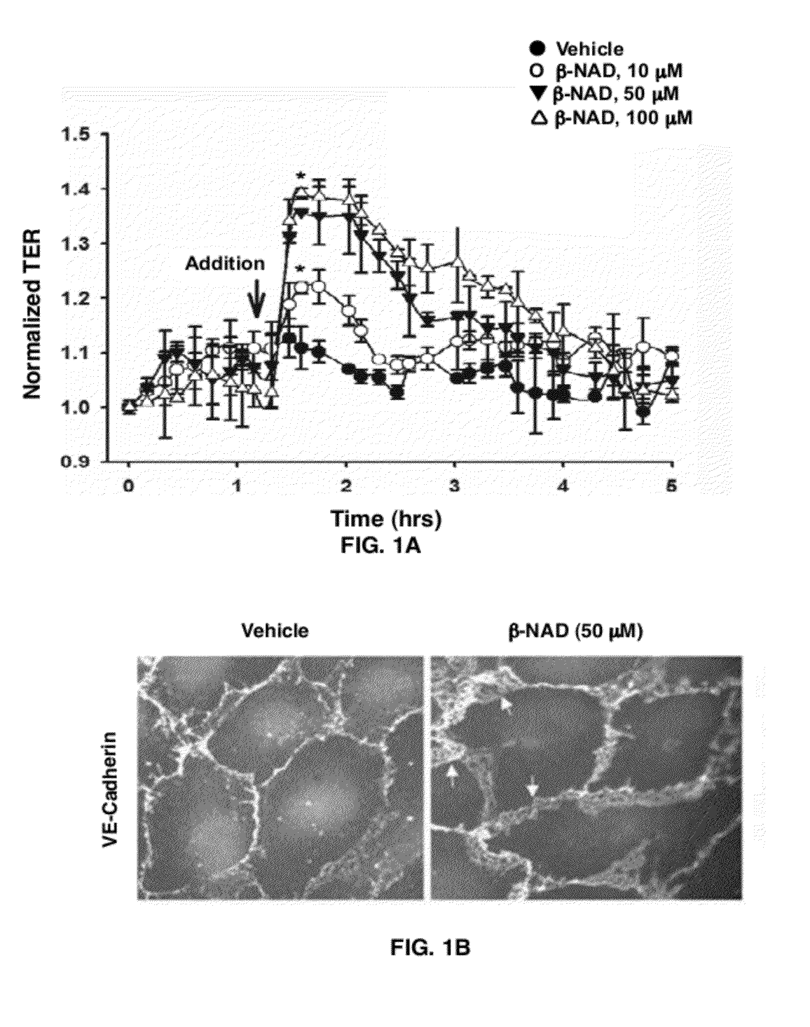

To examine β-NAD regulatation of endothelial monolayer integrity, β-NAD was used in the TER assay (FIG. 1). A dose-dependent effect of β-NAD on quiescent HPAEC monolayers was studied (FIG. 1A). There was a positive effect of micromolar concentrations of β-NAD on endothelial barrier function. β-NAD-treated HPAEC underwent changes in distribution of cell-cell junctional proteins, as demonstrated by immunofluorescence microscopy. VE-cadherin, a major component of endothelial adherens junctions, was more pronounced at the cellular periphery, presumably at cell-cell contacts (FIG. 1B). The calculated percentage of total cell surface area occupied by VE-cadherin-positive cell-cell junctions confirmed that β-NAD induced a significant increase in the surface area of cell-cell interfaces as a percentage of total cell surface area (FIG. 1C). Taken together, this data signify the role of β-NAD in the control of vascular permeability and maintaining a restrictive endothelial barrier.

### EXAMPLE 3

Expression of β-NAD-Activated P2Y Receptors in HPAEC and their Role in β-NAD-Induced TER Increase

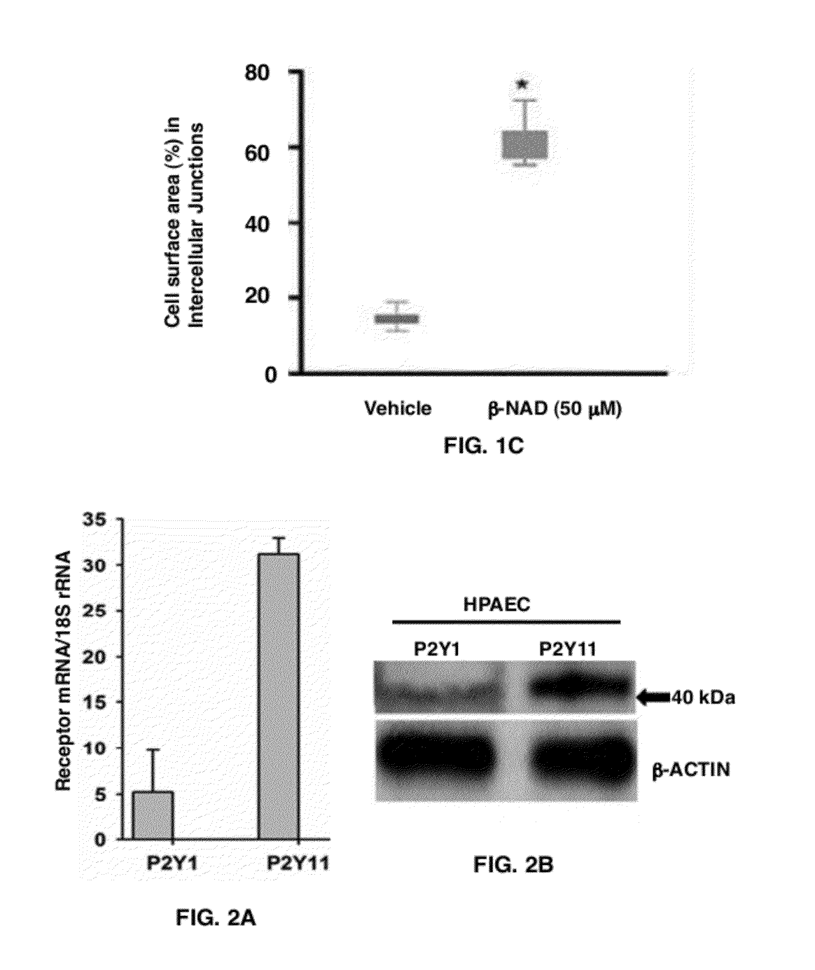

Extracellular β-NAD may activate the P2Y purine receptors P2Y1 and P2Y11. To evaluate the expression levels of these receptors in HPAEC, a semi-quantitative Real-Time RT-PCR analysis was carried out. HPAEC express both of these receptors (FIG. 2A) and the mRNA levels of P2Y11 receptor appears to be higher than P2Y1 receptor. Immunoblotting experiments with receptor specific antibodies indicate that HPAEC express both P2Y1 and P2Y11 receptor proteins (FIG. 2B). To reveal a possible involvement of either of them in HPAEC TER increase, two approaches were employed: (1) specific inhibition of the receptors by selective receptor antagonists and (2) specific depletion using siRNAs.

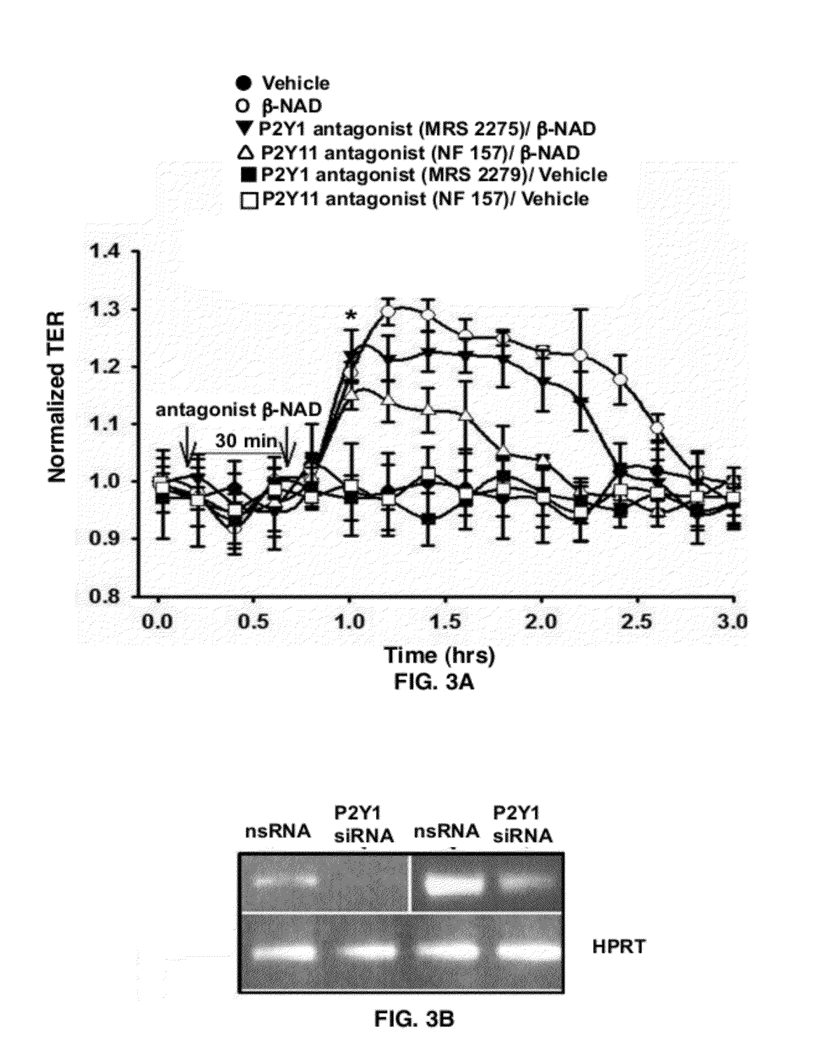

As shown in FIG. 3A, a treatment of HPAEC with either P2Y1 antagonist (MRS2279) or P2Y11 antagonist (NF157) attenuated the β-NAD-induced TER increase, suggesting involvement of these receptors in the enhancement of TER response. However, P2Y11 inhibition by NF157 attenuated the β-NAD-induced TER increase more significantly than P2Y1 inhibition by MRS2279. Data obtained may reflect the difference in the receptor expression levels and indicate the major role of highly expressed P2Y11 in HPAEC TER increase.

To confirm the inhibitory analysis results, P2Y1 and P2Y11 receptors were individually depleted using receptor-specific siRNAs and the depletion of both P2Y1 and P2Y11 receptor mRNAs were confirmed by RT-PCR analysis (FIG. 3B). The ECIS data (FIG. 3C, 3D) indicated that depletion of either P2Y1 or P2Y11 attenuated the β-NAD-induced HPAEC TER increase. However, the effect of depletion of the P2Y11 receptor on TER response was more profound (FIG. 3C). The control siRNA with scrambled sequence failed to attenuate the β-NAD-induced TER increase. These results suggest an involvement of P2Y1 and PY11, receptors in β-NAD-induced HPAEC TER increase.

### EXAMPLE 4

### Effects of β-NAD on Thrombin-, Lipopolysaccharide (LPS)- or Pneumolysin (PLY)-Induced HPAEC Barrier Dysfunction

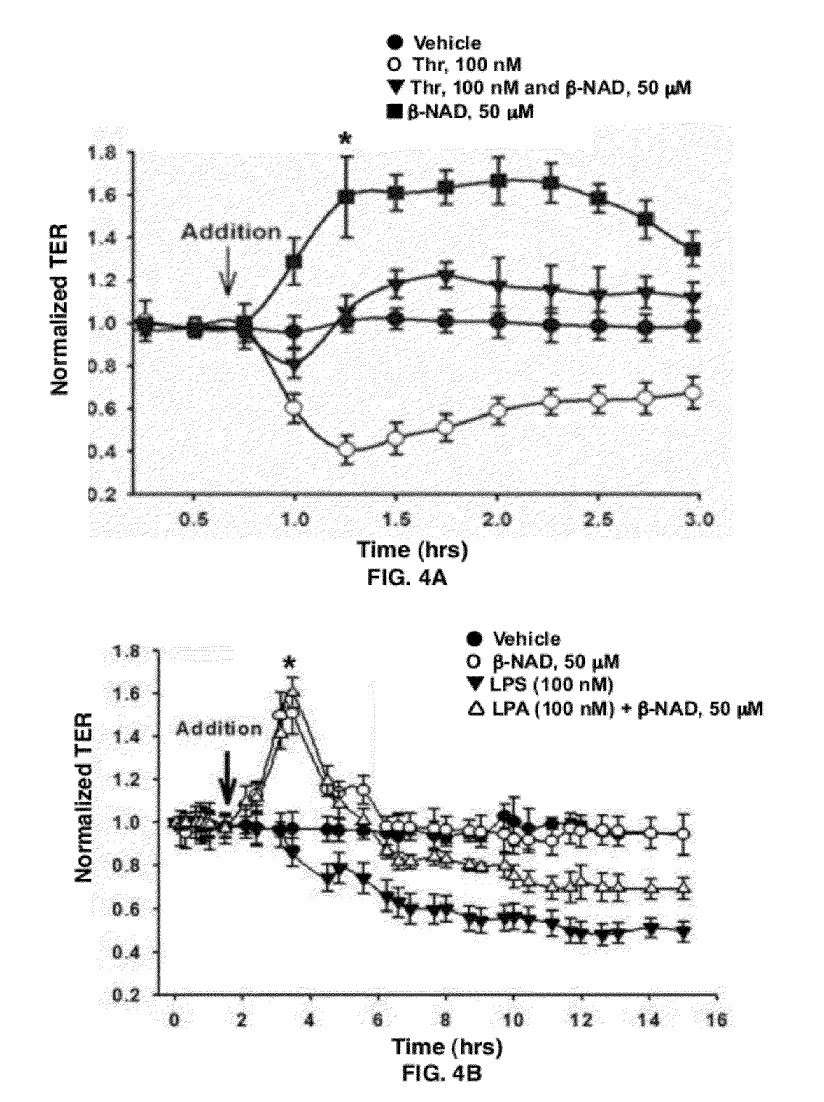

To evaluate endothelial cell barrier-protective functions of β-NAD, the effect of β-NAD treatment was analyzed on HPAEC challenged with various barrier-disruptive factors, such as protease thrombin, Gram-negative bacterial toxin LPS or Gram-positive bacterial toxin PLY. Thrombin, a protease activated on the surface of injured endothelium, stimulates protease-activated receptors (PARs) coupled to heterotrimeric G12/13, Gq/11 and Gi proteins which, in turn, stimulate PLCb, PKCa and RhoA pathways and inhibit adenylate cyclase (AC). This can eventually lead to activation of MLC kinase and inhibition of MLC phosphatase, stress fiber formation and endothelial cell barrier dysfunction. β-NAD-dependent cell signaling can antagonize thrombin-activated cascades. Simultaneous treatment of the cells with thrombin and β-NAD significantly attenuated the thrombin-induced endothelial cell permeability, demonstrating the barrier-protective effect of β-NAD (FIG. 4A). LPS, a component of the outer membrane of Gram-negative bacteria, acts as an endotoxin and elicits a strong immune response. LPS has been used as a model endotoxin to induce barrier disruption in HPAEC.

LPS-treatment of human umbilical vein endothelial cells (HUVEC) decreased the activity of myosin light chain (MLC) phosphatase (MLCP), resulting in an increase in MLC phosphorylation followed by cell contraction and an increase in endothelial cell permeability. To evaluate the protective role of β-NAD in LPS-induced HPAEC barrier disruption, TER measurement assay was performed in the cell monolayers.

As shown in FIG. 4B, HPAEC exposed to LPS (100 ng/ml) caused a significant and sustained decrease in HPAEC TER, which reflects a significant endothelial cell barrier dysfunction (˜60% decrease in TER from the baseline). However, added to LPS, β-NAD significantly attenuated the LPS-induced barrier disruption (˜30% decrease in TER from base line). These differences in TER values are significant as the protection is sustained for several hours. Treatment with β-NAD alone caused a significant initial increase in TER which is in full agreement with FIG. 1A. Similar results were also obtained when HPAEC was exposed to the PLY (125 ng/ml) (FIG. 4C). PLY is a pore-forming protein with multiple effects on eukaryotic cells. One of the effects characteristic for PLY-treated cells includes cytoskeletal reorganization due to elevation of intracellular Ca²⁺ followed by activation of Rho/Rho-kinase pathway. In order to test protective properties of β-NAD, HPAEC were treated with either PLY alone or in a mixture with 50 mM β-NAD, then measured TER response. FIG. 4C demonstrates a rapid loss of the monolayer integrity after PLY addition (˜80% decrease in TER from the baseline). In contrast, PLY added to the cells in a mixture with β-NAD, failed to produce such drastic effect (FIG. 4C), likely, because of rapid activation of interfering pathways leading to inhibition of RhoA and activation of MLCP.

### EXAMPLE 5

Role of Actin Cytoskeleton in_NAD-Dependent Cytoskeletal Rearrangement

Rho family GTPases are regulators of the actin cytoskeleton and influence the shape and movement of the cells. A major function of the Rho GTPases is reorganization of the actin cytoskeleton in response to various extracellular stimuli and the GTP-bound form of Rac1 has several common downstream targets that regulate the actin cytoskeleton and advance the motility of fibroblasts. Rho family GTPases, which are key regulators of cell migration, affect microtubules. Therefore, the dynamic cytoskeletal component(s) (actin and/or microtubules) indispensable for a β-NAD-induced increase in TER were identified. For these experiments, the cells were treated with cytoskeleton-disrupting agents prior to β-NAD stimulation.

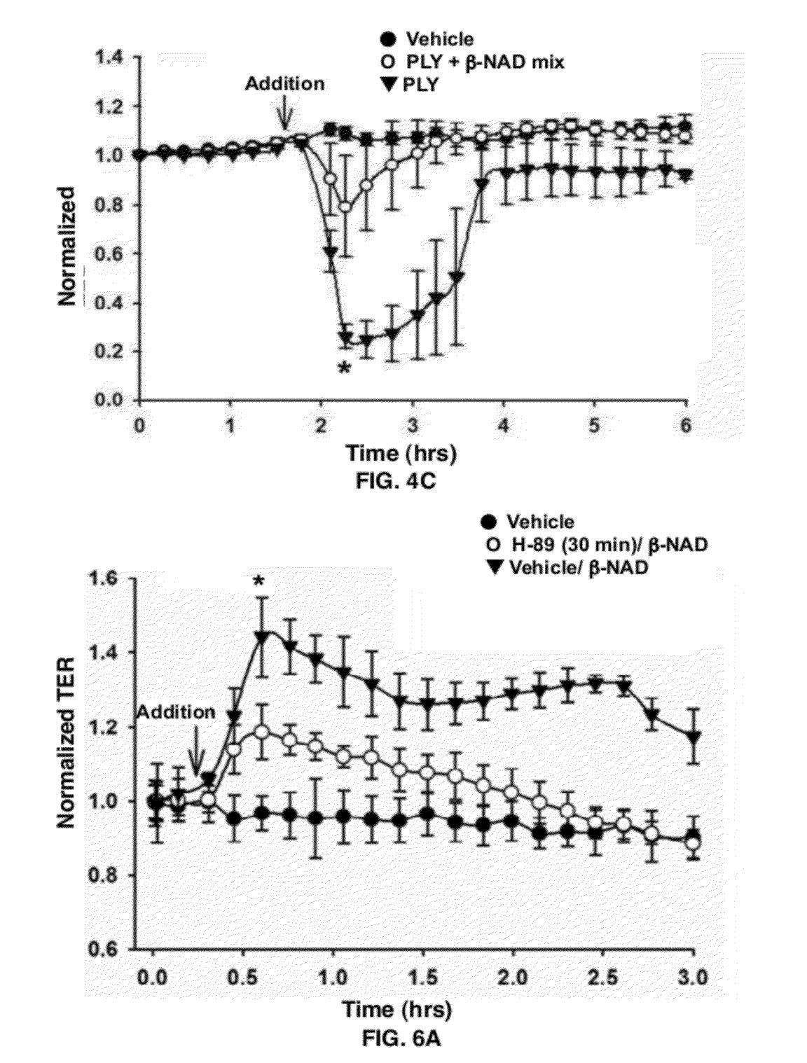

Using an ECIS approach, the involvement of the actin and tubulin components of the cytoskeleton in β-NAD-stimulated endothelial barrier enhancement were evaluated (FIGS. 5A-5B). First, the HPAEC monolayers were pretreated with the actin-depolymerizing agent, cytochalasin B, which produced a prompt attenuation in TER (FIG. 5A). Distinct from the protective effect observed for thrombin-induced barrier disruption (FIG. 4A), β-NAD treatment did not increase the TER when added after cytochalasin B. This data suggests a critical requirement for cytoskeletal rearrangement and an intact actin cytoskeleton in β-NAD-induced increase in HPAEC TER. Second, the microtubule-disrupting agent, nocodazole, compromises endothelial cell barrier integrity, was used. In contrast to the experiment with cytochalasin B, FIG. 5B demonstrates that β-NAD treatment restored endothelial cell barrier integrity disrupted by nocodazole. Thus, there is a pivotal role of actin filaments in dynamic cytoskeleton rearrangement induced by β-NAD. Moreover, β-NAD-dependent cell signaling might lead to regulation of the actin cytoskeleton via shifting the regulatory myosin light chain to dephosphorylated form (FIG. 5C) as was demonstrated for ATP-dependent endothelial cell barrier enhancement. Treatment of HPAEC with LPS caused a robust phosphorylation of MLC, which was significantly inhibited by β-NAD suggesting the involvement of MLCP in β-NAD-induced increase in TER (FIG. 5C).

### EXAMPLE 6

### Signaling Pathways for β-NAD-Induced Enhancement of EC Barrier Function

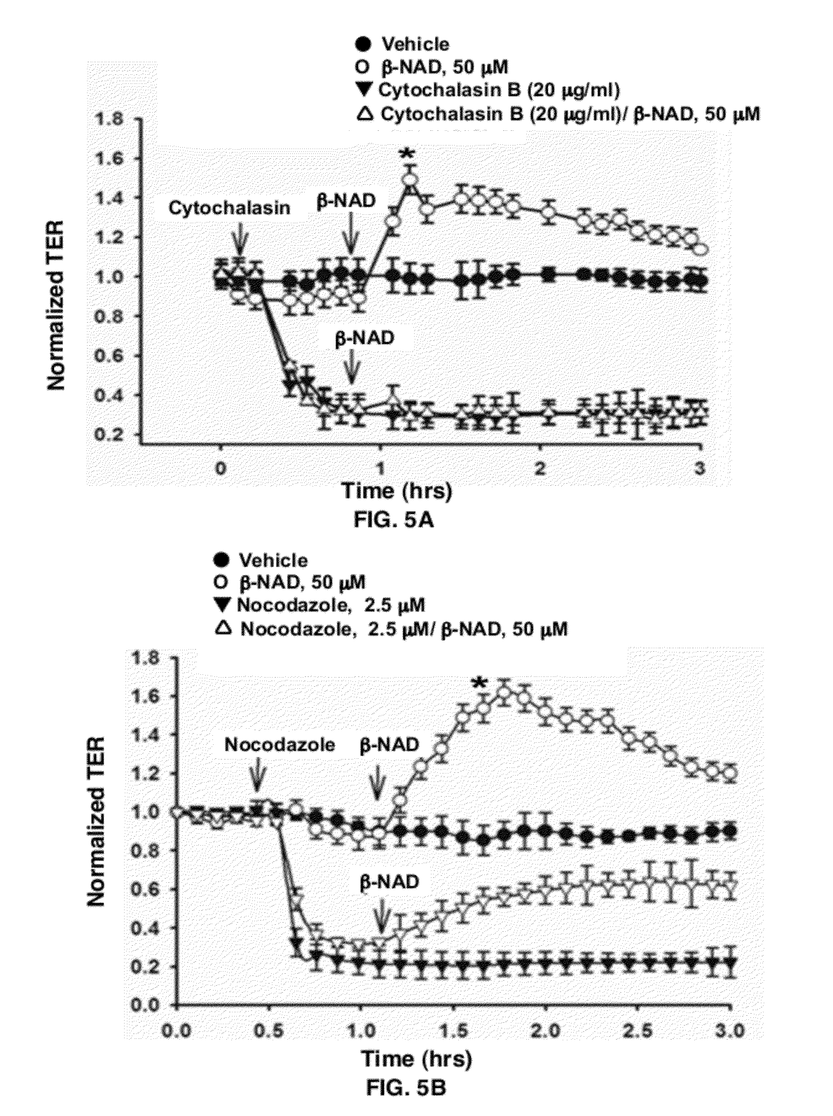

To elucidate the signaling pathways involved in β-NAD-induced HPAEC TER increase, the cAMP-activated protein kinase A (PKA) and the nucleotide exchange protein directly activated by cAMP (EPAC) pathways were examined. Since activation of P2Y11 receptors may lead to the Gas-mediated pathway including direct stimulation of adenylate cyclase, elevation of cAMP levels and cAMP-dependent activation of PKA, a simple inhibitory test was performed to confirm an activation of PKA and its participation in β-NAD-induced HPAEC barrier enhancement. For this test, H-89, an inhibitor of PKA activity, was used in ECIS experiments. HPAEC were pre-treated with H-89 for 30 min and then challenged with β-NAD and the effect of β-NAD-mediated barrier enhancement was determined using TER measurement. FIG. 6A indicates that H-89 pre-treatment attenuated the β-NAD-induced HPAEC TER increase.

Another cAMP-dependent signaling cascade, EPAC1/Rap1/Rac1 may also be involved in endothelial cell barrier protection. To elucidate whether or not EPAC1 is also critical for β-NAD-inducible TER response, the expression of EPAC1 in HPAEC was depleted with the siRNA specific for EPAC1 (FIG. 6B) and then the cells were challenged with β-NAD in TER assay (FIG. 6C). HPAEC with depleted EPAC1 have markedly decreased TER response to β-NAD signifying the involvement of EPAC1 in β-NAD-induced TER response. Small GTPases of the Rho family regulate many aspects of cytoskeletal dynamics and three members of the family (Rac1, RhoA and Cdc42) have been studied. Rho family GTPases control cell growth, cytokinesis, cell motility, trafficking and organization of the cytoskeleton. Rac1 could be involved in β-NAD-induced increase in TER as a downstream target of the EPAC1 pathway. To prove this, the HPAEC monolayers were treated with β-NAD, and the cell lysates obtained at several time points of β-NAD stimulation were used to determine the levels of Rac1 activation by G-LISA assay. Data shown in FIG. 6D demonstrate dramatic β-NAD-dependent activation of Rac1 at early time points and the activity gradually returned to the basal values by 30 min. Time-dependent increase of Rac1 activation corroborates with the rapid increase in TER of HPAEC upon β-NAD treatment (FIG. 1A).

β-NAD significantly increases the TER of pulmonary endothelial cells in a dose-dependent manner (FIG. 1A) and attenuates the thrombin-, LPS-, and PLY-induced EC barrier disruption (FIGS. 4A-4C). β-NAD induces rearrangement of VE-cadherin suggesting tightening of cell-cell contacts leading to barrier enhancement (FIG. 1B). These results demonstrate that β-NAD is an extracellular nucleotide in the regulation of endothelial permeability.

Although β-NAD structure is similar to those of the classic ligands of purine receptors, ATP and ADP, it is a ligand of purine receptors. Interactions of β-NAD can bind to two purine receptors, P2Y1 and P2Y11. Such selectivity indicates that extracellular β-NAD could be an attractive, physiologically relevant agent for positive regulation of endothelial barrier function, since these receptors are coupled only to heterotrimeric Gs and Gq proteins. Indeed, Gs protein is a well-known direct activator of AC, and elevation of cAMP levels in endothelial cells essentially leads to an enhancement of barrier integrity. Activation of heterotrimeric Gq protein is followed by direct activation of the phospholipase Cb and, therefore, elevation of inositol 1,4,5-triphosphate (IP₃) and diacylglycerol (DAG) levels. These two second messengers stimulate, in turn, intracellular calcium elevation, activation of PKC and/or PKG pathways. In contrast, ATP and ADP interact with four different P2Y receptors and can also activate Gi protein, an inhibitor of AC.

The present invention demonstrates that β-NAD serves as an effector of endothelial integrity. The experiments with stimulated HPAEC monolayers revealed that β-NAD is a strong positive regulator of endothelial integrity. HPAEC were used because they express both β-NAD-activated purine receptors and one can evaluate their involvement in β-NAD-dependent barrier enhancement. Inhibitory analysis based on receptor-selective antagonists and sequence-specific siRNAs showed that both P2Y1 and P2Y11 receptors are involved in β-NAD-induced endothelial cell response. β-NAD-activated receptors stimulate cAMP synthesis followed by activation of two cAMP-dependent pathways, PKA and EPAC1/Rac1. Both of them likely led to actin cytoskeleton rearrangement via RhoA/Rho-kinase inhibition and activation of MLCP. The actin component of cytoskeleton played an indispensable role in the HPAEC monolayer integrity enhancement, while microtubules were not involved in the TER response induced by β-NAD. Taken together, activation of both P2Y receptors lead to actin reorganization and barrier protection/enhancement, although via at least two different signaling pathways. In HPAEC treated with β-NAD, Gas-induced stimulation of AC leads to two cAMP-dependent pathways, PKA and EPAC1 followed by rapid activation of Rac1. Thus, β-NAD is a very efficient regulator of endothelial integrity as shown by the experiments with various endothelial cell barrier-disruptive factors such as thrombin, and the bacterial toxins LPS and PLY. In summary, β-NAD is protective against thrombin, LPS- and PLY-induced endothelial cell barrier dysfunction via cAMP-activated PKA and EPAC1/Rac1-dependent actin cytoskeleton rearrangement.

The present invention demonstrates a mechanism of β-NAD-mediated rapid and dose dependent increase in transendothelial electrical resistance (TER) of the pulmonary endothelial cell barrier. β-NAD attenuates both Gram positive (pneumylysin, PLY) and Gram negative (lipopolysaccharide, LPS)-induced EC barrier dysfunction in human pulmonary artery endothelial cells. Therefore, b-NAD-mediated endothelial activation of P2Y1/P2Y11 receptors signaling protects the lung vascular barrier against acute lung injury in sepsis-induced lung inflammation in vivo.

To test this, a murine model of ALI induced by intratracheal administration of LPS was used. β-NAD (50 μM final blood concentrations) attenuated the inflammatory response with a decreased accumulation of cells and protein in bronchioalveolar lavage (BAL) and reduced neutrophil infiltration and extravasation of Evans blue dye (EBD)-albumin into the lung tissue. In addition, the histological examination demonstrated fewer neutrophils in the pulmonary interstitium and decreased interstitial edema in the b-NAD treated specimens. Quantitative real-time PCR data demonstrated that b-NAD inhibits the expression of pro-inflammatory cytokines and activates anti-inflammatory cytokines. Further, a 15 day study of the mortality of LPS vs. LPS/β-NAD treated mice indicated that the β-NAD treated mice demonstrated significantly reduced morality compared to LPS only treated mice. These findings suggest that β-NAD exerts a protective role against ALI/ARDS in vivo.

### EXAMPLE 7

### β-NAD Reduces Pulmonary Vascular Endothelial Barrier Dysfunction and Lung Inflammation in LPS Treated Mice

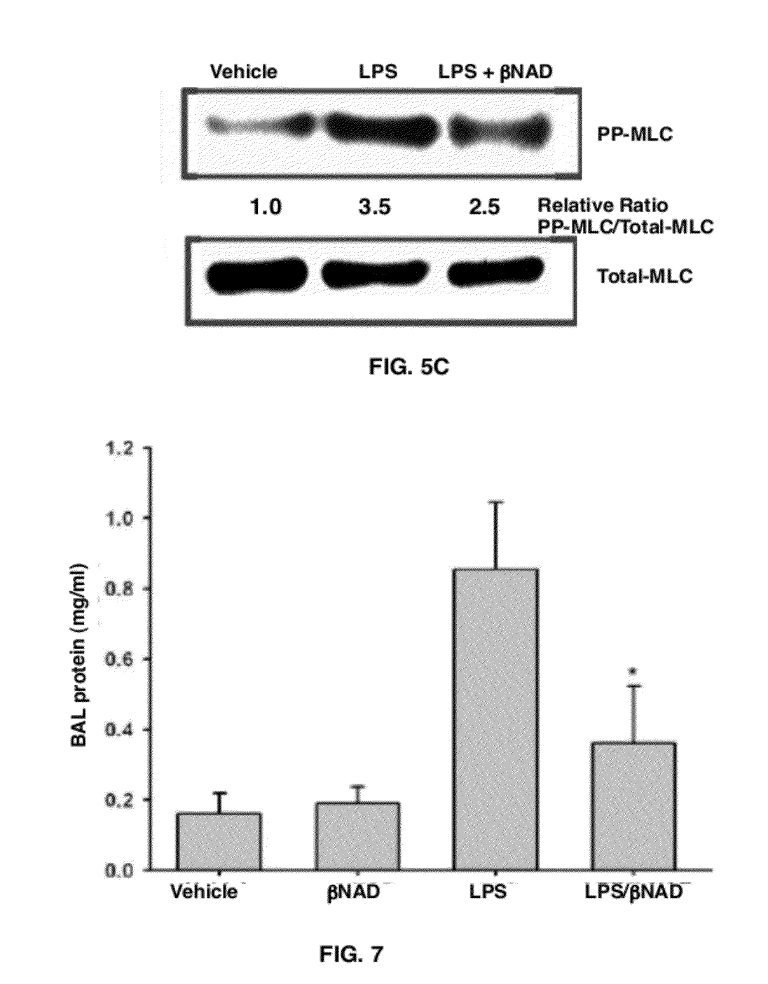

Mice challenged with LPS for 18 hr significantly increased the pulmonary BAL protein concentration compared to mice given saline or β-NAD alone. This increase in LPS-induced BAL protein accumulation was significantly attenuated when mice were treated simultaneously with β-NAD (i.v.) and LPS (i.t.) suggesting the protective role of β-NAD (FIG. 7).

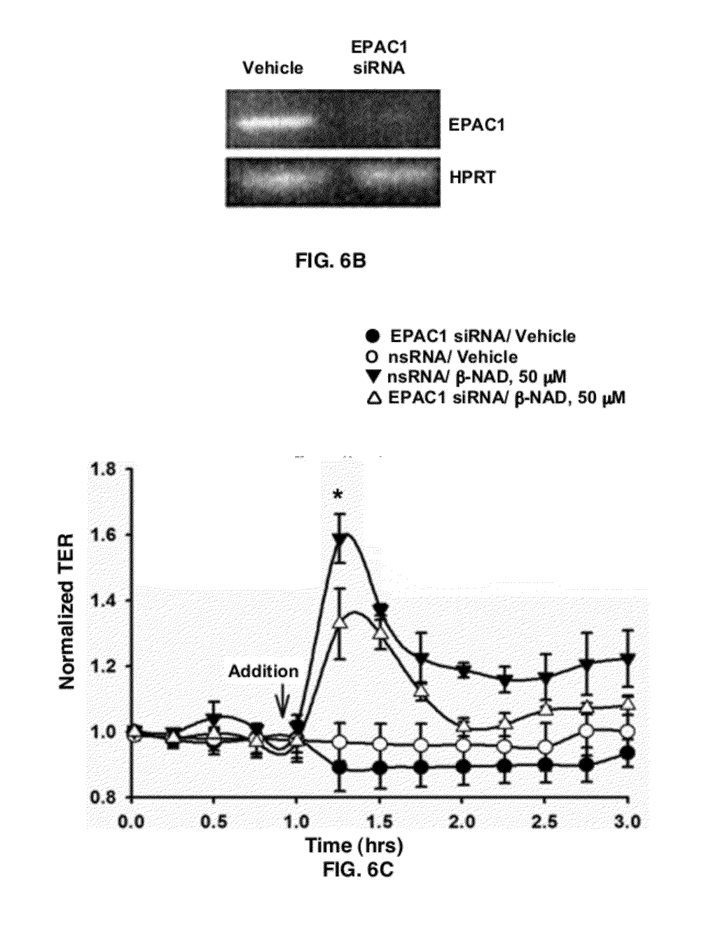

LPS challenge also induced pulmonary edema as evidenced by extravasation of Evans Blue Dye (EBD)-albumin into the lung parenchyma. Challenge with saline or b-NAD alone minimally altered the levels of Evans Blue Dye leakage compared to LPS exposure alone that significantly increased levels of Evans Blue Dye-albumin (FIG. 8A). The level of EBD-albumin was attenuated by b-NAD in the LPS treated mice (FIG. 8A). The measurement of EBD-albumin in the LPS treated mice averaged 16.81 μg/g while the b-NAD/LPS average was 10.41 μg/g and the β-NAD/saline control was 6.54 μg/g and vehicle was 4.57 μg/g, (FIG. 8A). A gross view of the left lung was photographed showing the LPS/PBS lung to be consistent with the increase in EBD leakage and decreased extravasation in the LPS/R-NAD treated lung (FIG. 8B).

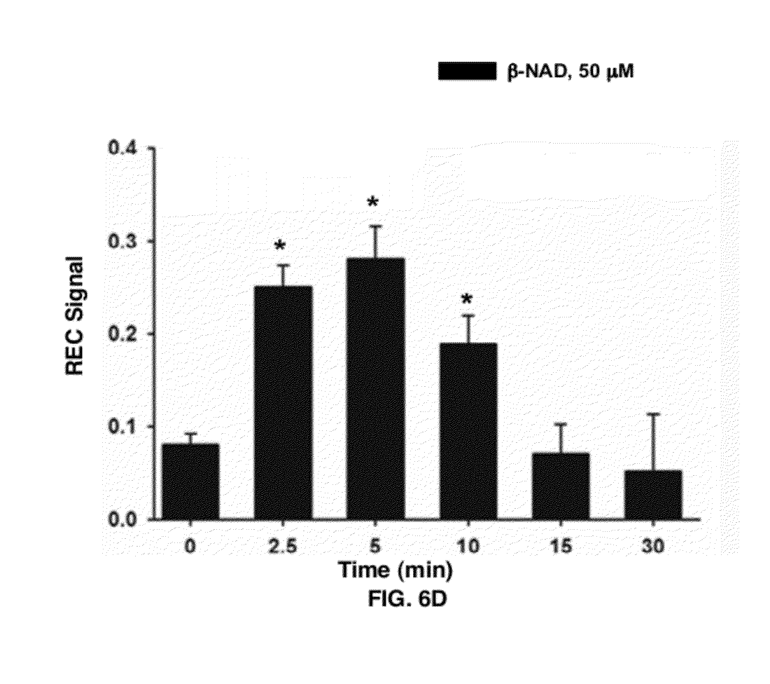

The white blood cell (WBC) count was consistent with the protein and Evans Blue Dye albumin results as a quantitative microscopic assessment of the cell count of BAL fluid on hemocytometer showed that control lungs contained few neutrophils, LPS treatment led to an increased number of neutrophils and the LPS/b-NAD treated mice demonstrated a decrease in neutrophil count when compared to the LPS only treated mice (FIG. 9).

### EXAMPLE 8

Histology Demonstrated that β-NAD Decreased LPS Induced Lung Inflammation

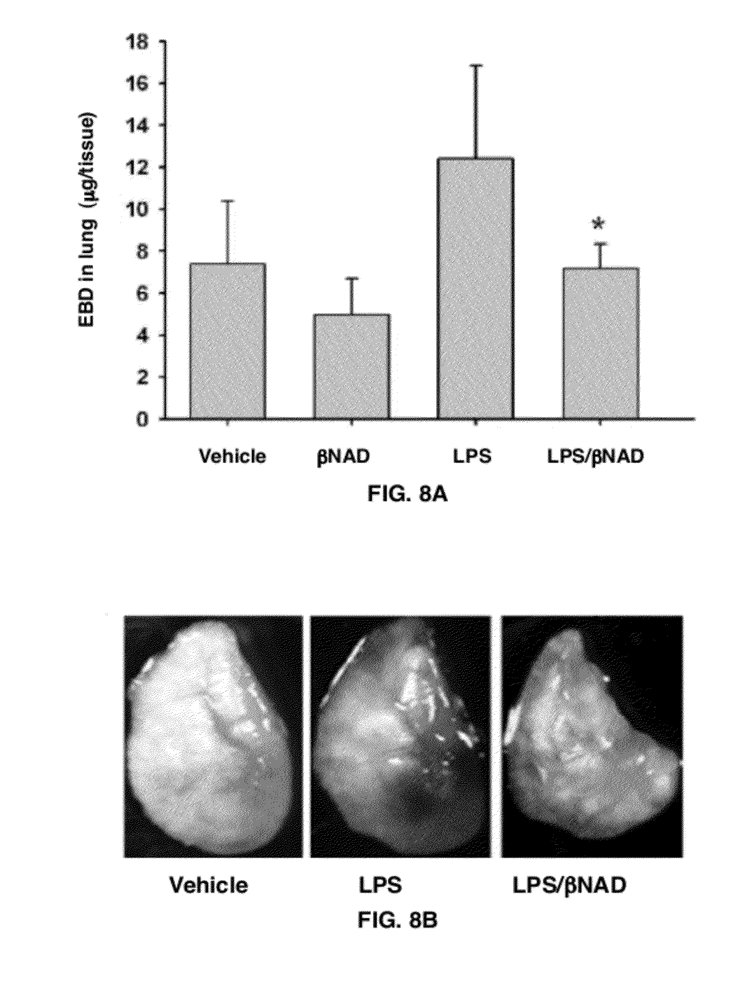

Mice challenged with LPS for 18 hr demonstrated an inflammatory response typical for ALI/ARDS compared with saline treated controls (FIG. 10). Histological evaluation of the lung tissue displayed an increased interstitial edema and infiltration of neutrophils in the LPS treated lung that was less prominent in the LPS/β-NAD treated lung. The histology was heterogeneous as typically occurs in ALI. β-NAD alone has no effect.

### EXAMPLE 9

### β-NAD Decreases Mortality and Improves Animal Survival in LPS-Induced Lung Injury

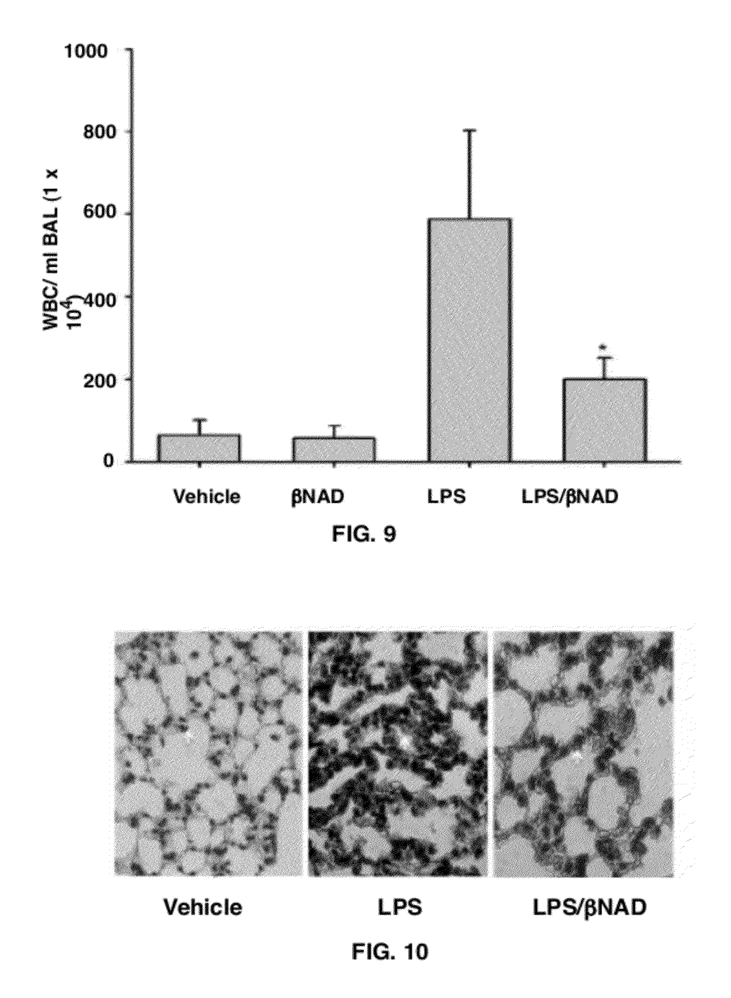

To evaluate whether β-NAD treatment protects the mice from LPS-induced lung injury, β-NAD was used during LPS challenge and post-treatment for two days. The mice given LPS either with or without b-NAD were symptomatic within hours with respiratory distress, lethargy and general malaise. The majority of mice treated with LPS/β-NAD recovered and lived longer, while the LPS alone challenged mice died within 4 days (FIG. 11). All LPS and LPS/b-NAD treated mice lost weight indicating a severe response to the LPS toxin. The LPS/β-NAD treated mice recovered and their behavior was significantly improved. β-NAD alone has no effect.

### EXAMPLE 10

### Effect of β-NAD Treatment on the Gene Expression of Cytokines

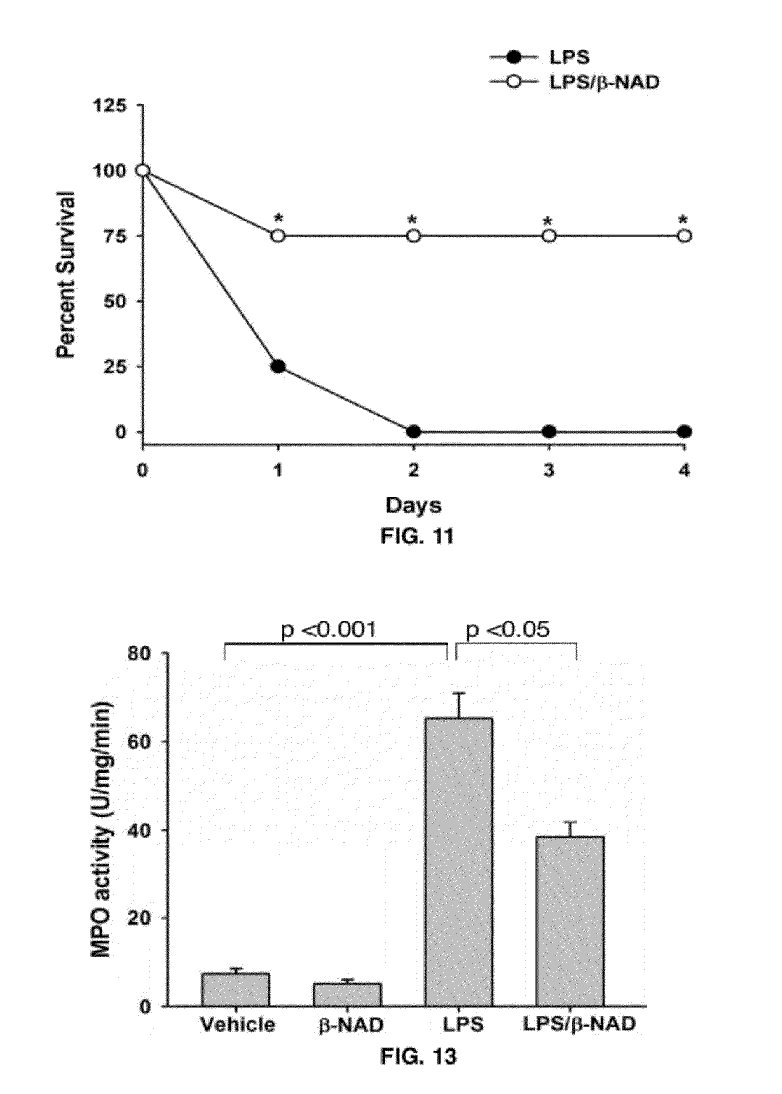

Real-time polymerase chain reaction (RT-PCR) results of pro-inflammatory and anti-inflammatory transcripts in LPS and LPS/β-NAD treated animals are shown in FIG. 12. The proinflammatory cytokines (IL-1α, IL-1β, IFN-γ and TNF-α) gene expressions were upregulated in the LPS only challenged mice group and their expression levels were substantially or markedly downregulated in the LPS/β-NAD mice group with /β-NAD treatment suggests the involvement of β-NAD in the attenuation of proinflammatory cytokines gene expression levels. The anti-inflammatory cytokines (IL-4, IL-10, and IL-13) gene expression levels were rare with little to no presence in the LPS alone challenged mice group, however, their expressions were elevated in the LPS/β-NAD treated mice group indicating barrier synthesis or inducement in the presence of β-NAD treatment.

### EXAMPLE 11

### β-NAD Attenuates LPS-Induced Myeloperoxidase Activity in Lungs

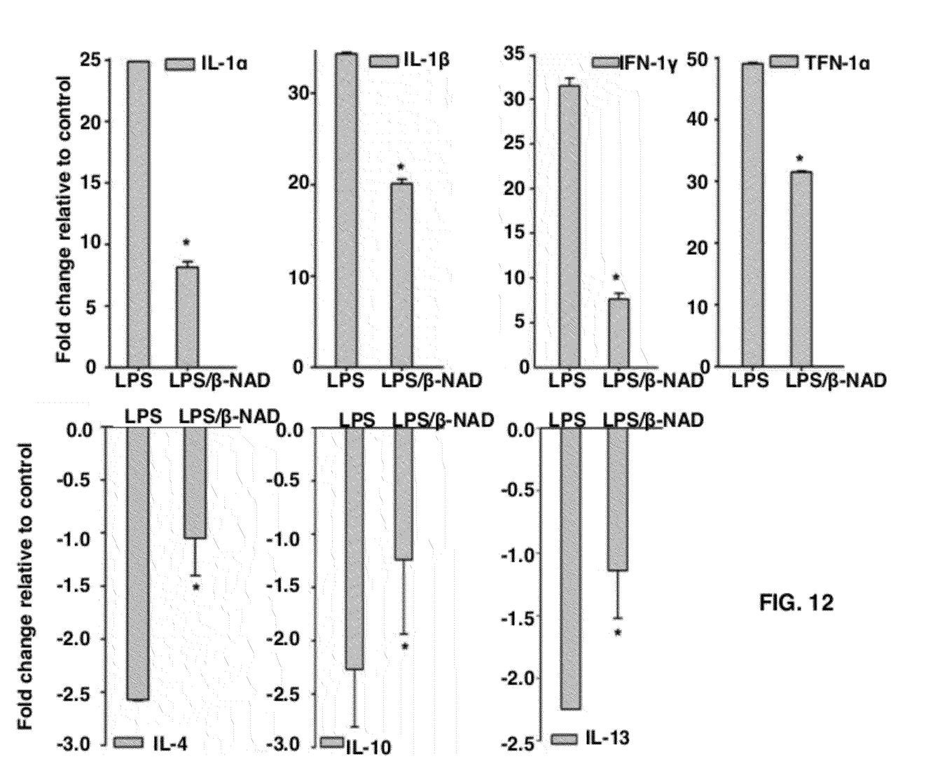

MPO (an index of neutrophil sequestration in the lungs) activity was measured in snap-frozen right lungs. MPO activity was increased significantly in LPS challenged mice. However, LPS/b-NAD-treated mice attenuated MPO activity (FIG. 13) suggesting b-NAD activated signaling mediated protection. In addition, immunohistochemistry data show significant amount of neutrophil sequestration (as evidence by increased MPO staining in LPS challenged mice,) that was attenuated in LPS/β-NAD (FIG. 13).

### EXAMPLE 12

Histology Demonstrates that β-NAD Decreased LPS-Induced Lung Inflammation.

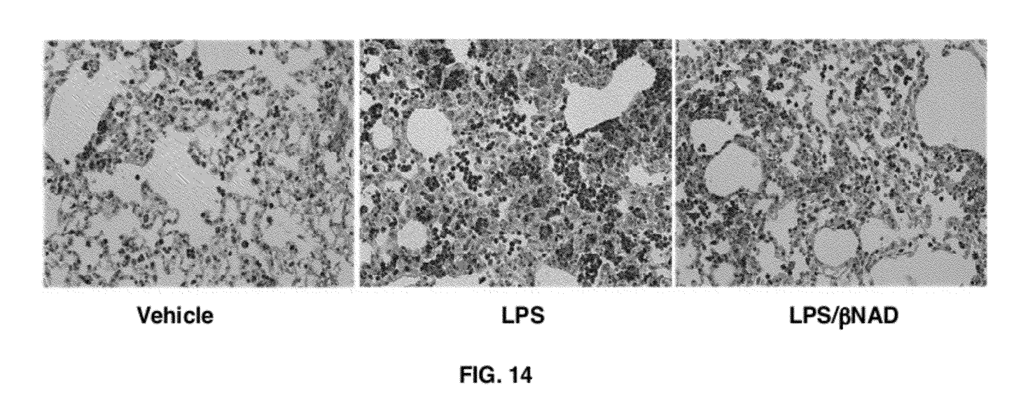

Mice challenged with LPS for 18 hr demonstrated an inflammatory response typical for ALI/ARDS compared with saline treated controls. Histological evaluation (FIG. 14) of the lung tissue displayed an increased interstitial edema and infiltration of neutrophils in the LPS treated lung that was less prominent in the LPS/β-NAD treated lung. However, the histology was noted to be heterogeneous as typically occurs in ALI. Histological specimens in the control mice displayed normal lung parenchyma and b-NAD alone treated mice also had no effect.

### Discussion

It is well known that endothelial hyperpermeability leads to increased pulmonary edema in ALI/ARDS. Acute lung injury is typified by pulmonary microvascular endothelial disarray with cellular breakdown and subsequent endothelial permeability and interstitial edema. The present invention demonstrates that β-NAD administration significantly attenuated the accumulation of protein in LPS-induced murine models of ALI and suggesting an improvement of endothelial cell barrier function via β-NAD-induced signaling. In addition, measurement of EBD-albumin extravasation into the lung parenchyma confirmed that LPS-induced albumin increase was also attenuated in the β-NAD treated mice. These results indicate that attenuation of vascular leak occurred in the β-NAD treated mice, and that the LPS only treated mice had an increase in protein and albumin leakage into the lung parenchyma. In TER measurement assays, LPS caused significant human pulmonary artery endothelial cells (HPAEC) barrier disruption and the addition of β-NAD to the cells significantly attenuated the LPS-induced barrier disruption.

Histological evaluation of the lung tissue displayed interstitial edema and increased neutrophils. This illustrates the endothelial barrier disruption that is known to occur with ALI/ARDS and the heterogeneity displayed is another feature that is known to occur complicating ventilator strategies in animal and human models of ALI/ARDS. VE-cadherin, a major component of the endothelial adherent junctions, was more pronounced at the cell periphery and increased the surface area of cell-cell interfaces. Histological examination of LPS challenged lung tissue showed morphological changes and β-NAD seems to attenuate these changes. Vehicle treated mice demonstrated minimal damage. Histological results displayed interstitial edema with increased edema and neutrophils, and the LPS/β-NAD treated specimens show improvement when compared to the LPS only specimens.

Murine lung injury induced by LPS is a model that has been shown to be consistent with sepsis induced acute lung injury. Injury is characterized by neutrophil infiltration into the lung within 24 hours with associated increased inflammatory mediators, interstitial edema and early mortality. These factors contribute to the oxidative stress and inflammatory response of the host. LPS-induced lung injury caused 100% mortality within four days. Murine mortality when given β-NAD simultaneously with LPS and then b-NAD twice a day for three days was significantly improved. In human medicine sepsis syndrome with multi-organ dysfunction remains the most common cause of death in patients with sepsis induced ARDS. LPS in murine induction of ARDS represents the model of sepsis with ARDS with pulmonary endothelial cell barrier disruption as the fundamental pathology. Gram-negative sepsis is a very common cause of ALI/ARDS.

LPSβ-NAD treated mice had lower expression levels of the pro-inflammatory cytokines with an increased anti-inflammatory cytokines gene expression in the LPS/β-NAD treated mice lungs compared to the LPS only treated mice which showed very high levels of pro-inflammatory cytokines gene expression and less or no expression of anti-inflammatory cytokines gene expression. This suggests an attenuation of the destructive inflammatory process in the β-NAD treated mice.

The gene expression levels of anti-inflammatory cytokines (IL-4, IL-10 and IL-13) were elevated in the mice treated with LPS/β-NAD. An IL-1 pro-inflammatory cytokine not measured was IL-1 receptor antagonist (IL-1ra), a cytokine that is also stimulated by LPS. However, in the lung the synthesis of IL-1ra is known to be inadequate and this permits increased damage of the lung in ARDS. The elevation of anti-inflammatory cytokines indicates that the anti-inflammatory mechanism in the β-NAD treated mice was improved over the LPS only treated mice. There was a significant increase in mice survival of the β-NAD treated mice the elevation of IL-10 could be an indicator of the improved mortality seen as there may be an association between increased mortality rates and decreased concentrations of IL-10.

Thus, the present invention demonstrates in vitro and in vivo that β-NAD attenuates the endothelial cell barrier dysfunction evidenced by decreased TER in vitro; decreased protein leak, EBD extravasation, and white blood cell count in BAL in vivo. Gross observation of the lung and microscopic histological evaluation is consistent with these results.

One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for treating inflammation in the lungs of a subject in need of such treatment, comprising the step of:
administering an effective amount of a composition comprising beta-nicotinamide adenine dinucleotide to said subject.

2. The method of claim 1, wherein the inflammation is induced by or associated with elevated levels of one or more cytokines in the lungs of the subject.

3. The method of claim 2, wherein the inflammation is induced by or associated with elevated levels of interleukin-1alpha, interleukin-1beta, interleukin-8, interleukin-17, TNF-alpha, interferon-gamma or tissue growth factor beta.

4. The method of claim 1, wherein the inflammation is induced by or associated with increased levels of one or more inflammatory cells in the lungs.

5. The method of claim 4, wherein the one or more inflammatory cells are eosinophils, lymphocytes, macrophages, neutrophils or monocytes.

6. The method of claim 1, wherein the inflammation is associated with asthma, allergic asthma, infection, emphysema, inflammatory lung injury, bronchiolitis obliterans, pulmonary sarcoisosis, chronic obstructive pulmonary disease, interstitial lung disease, idiopathic pulmonary fibrosis, adult respiratory distress syndrome, bronchiectasis, lung eosinophilia, interstitial fibrosis, acute lung injury, sepsis, inflammation mediated lung cancer, or cystic fibrosis.

7. The method of claim 1, wherein the inflammation is associated with transplantation of an organ, tissue and/or cells to the subject.

8. The method of claim 1, wherein administration of said composition elevates levels of an anti-inflammatory cytokine in the lungs of the subject.

9. The method of claim 8, wherein the anti-inflammatory cytokine is interleukin-4, interleukin-13 or interleukin-10.

10. The method of claim 1, wherein administration of said composition reduces levels of a pro-inflammatory cytokine in the lungs of the subject.

11. The method of claim 10, wherein the pro-inflammatory cytokine is interleukin-1alpha, interleukin-1beta, interleukin-8, interleukin-17, TNF-alpha, interferon-gamma or tissue growth factor beta.

12. The method of claim 1, wherein administration of said composition results in an average minimum plasma concentration of beta-nicotinamide adenine dinucleotide that is greater than about 100 mM in the plasma of the subject.

13. The method of claim 1, wherein administration of said composition results in an average maximum concentration of beta-nicotinamide adenine dinucleotide that is less than 100 mM in the plasma of the subject.

14. The method of claim 1, wherein said composition is administered systemically, orally, intravenously, intramuscularly, subcutaneously, intraorbitally, intranasally, intracapsularly, intraperitoneally, intracisternally, intratracheally, intraarticularly or by absorption through the skin.

15. The method of claim 1, further comprising:
administering an anti-inflammatory agent, bronchodilator or an antibiotic.

16. A method for treating a pulmonary disorder in a subject in need of such treatment, comprising the steps of:
administering an effective amount of a composition comprising beta-nicotinamide adenine dinucleotide to said subject, wherein administration of said composition results in an average minimum plasma concentration of beta-nicotinamide adenine dinucleotide that is greater than 100 mM in the plasma of the subject and an average maximum concentration of beta-nicotinamide adenine dinucleotide that is less than 100 mM in the plasma of the subject; and
administering a therapeutic agent selected from the list consisting of an anti-inflammatory agent, bronchodilator and an antibiotic.

17. The method of claim 16, wherein administration of said composition elevates levels of an anti-inflammatory cytokine in the lungs of the subject, reduces levels of a pro-inflammatory cytokine in the lungs of the subject, or elevates levels of an anti-inflammatory cytokine and reduces levels of a pro-inflammatory cytokine in the lungs of the subject.

18. A method for increasing integrity of a vascular barrier in a subject, comprising the step of:
contacting one or both of human P2Y1 receptors or P2Y11 receptors in the subject with an amount of a composition comprising beta-nicotinamide adenine dinucleotide effective to activate said receptors; wherein activation thereof increases the integrity of the vascular barrier in the subject.

19. The method of claim 18, wherein a pulmonary disorder in the subject has reduced the integrity of the vascular barrier.

20. The method of claim 19, wherein the pulmonary disorder is asthma, allergic asthma, infection, emphysema, inflammatory lung injury, bronchiolitis obliterans, pulmonary sarcoisosis, chronic obstructive pulmonary disease, interstitial lung disease, idiopathic pulmonary fibrosis, adult respiratory distress syndrome, bronchiectasis, lung eosinophilia, interstitial fibrosis, acute lung injury, sepsis, inflammation mediated lung cancer, or cystic fibrosis.


SEQUENCE LISTING (24 sequences)

SEQ ID NO: 1 - DNA, 24 bp, artificial sequence
  forward primer for P2Y1
tattcatcatcggcttcctgggca

SEQ ID NO: 2 - DNA, 26 bp, artificial sequence
  reverse primer for P2Y1
agcggcatctccgtgtacatgttcaa

SEQ ID NO: 3 - DNA, 23 bp, artificial sequence
  forward primer for P2Y11
ctcctatgtgccctaccacatca

SEQ ID NO: 4 - DNA, 25 bp, artificial sequence
  reverse primer for P2Y11
agctttgcagacatagcccaggcca

SEQ ID NO: 5 - DNA, 24 bp, artificial sequence
  forward primer for EPAC1
ttgttgtcaacccacacgaagtgc

SEQ ID NO: 6 - DNA, 24 bp, artificial sequence
  reverse primer for EPAC1
gaggccaaacatgacggcaaagaa

SEQ ID NO: 7 - DNA, 21 bp, artificial sequence
  forward primer for GAPDH
catggcctccaaggagtaaga

SEQ ID NO: 8 - DNA, 21 bp, artificial sequence
  reverse primer for GAPDH
gagggagatgctcagtgttgg

SEQ ID NO: 9 - DNA, 21 bp, artificial sequence
  forward primer for IL-1alpha
gcaccttacacctaccagagt

SEQ ID NO: 10 - DNA, 21 bp, artificial sequence
  reverse primer for IL-1alpha
aaacttctgcctgacgagctt

SEQ ID NO: 11 - DNA, 22 bp, artificial sequence
  forward primer for IL-1beta
gcaactgttcctgaactcaact

SEQ ID NO: 12 - DNA, 21 bp, artificial sequence
  reverse primer for IL-1beta
atcttttggggtccgtcaact

SEQ ID NO: 13 - DNA, 20 bp, artificial sequence
  forward primer for IL-4
ggtctcaacccccagctagt

SEQ ID NO: 14 - DNA, 23 bp, artificial sequence
  reverse primer for IL-4
gccgatgatctctctcaagtgat

SEQ ID NO: 15 - DNA, 23 bp, artificial sequence
  forward primer for IL-6
tagtccttcctaccccaatttcc

SEQ ID NO: 16 - DNA, 21 bp, artificial sequence
  reverse primer for IL-6
ttggtccttagccactccttc

SEQ ID NO: 17 - DNA, 21 bp, artificial sequence
  forward primer for IL-10
gctcttactgactggcatgag

SEQ ID NO: 18 - DNA, 20 bp, artificial sequence
  reverse primer for IL-10
cgcagctctaggagcatgtg

SEQ ID NO: 19 - DNA, 19 bp, artificial sequence
  forward primer for IL-13
cctggctcttgcttgcctt

SEQ ID NO: 20 - DNA, 21 bp, artificial sequence
  reverse primer for IL-13
ggtcttgtgtgatgttgctca

SEQ ID NO: 21 - DNA, 21 bp, artificial sequence
  forward primer for IFN-gamma
atgaacgctacacactgcatc

SEQ ID NO: 22 - DNA, 21 bp, artificial sequence
  reverse primer for IFN-gamma
ccatccttttgccagttcctc

SEQ ID NO: 23 - DNA, 23 bp, artificial sequence
  forward primer for TNF-alpha
ccctcacactcagatcatcttct

SEQ ID NO: 24 - DNA, 19 bp, artificial sequence
  reverse primer for TNF-alpha
gctacgacgtgggctacag